(19) United States
(12) Patent Application Publication
(10) Pub. No.: US 2023/0347075 A1
(43) Pub. Date: Nov. 2, 2023

(54) FLOW RESTRICTOR FOR DRUG DELIVERY DEVICE

(71) Applicant: AMGEN INC., Thousand Oaks, CA (US)

(72) Inventors: Ali Nekouzadeh, Thousand Oaks, CA (US); Mehran Mojarrad, Thousand Oaks, CA (US); Joshua Tamsky, Thousand Oaks, CA (US); Paul Daniel Faucher, Thousand Oaks, CA (US); Scott R. Gibson, Thousand Oaks, CA (US); Kimberly Sepulveda, Thousand Oaks, CA (US); Sheldon B. Moberg, Thousand Oaks, CA (US)

(21) Appl. No.: 17/780,596

(22) PCT Filed: Nov. 17, 2020
(86) PCT No.: PCT/US2020/060867

§ 371 Date: May 27, 2022

Related U.S. Application Data
(60) Provisional application No. 62/944,083, filed on Dec. 5, 2019.

(51) Int. Cl. A61M 5/48; A61M 5/158; A61M 5/315

(52) U.S. Cl. CPC A61M 5/482; A61M 5/158; A61M 5/31576

(57) ABSTRACT

A drug delivery device includes a housing, a container disposed in the housing, a drive mechanism, a needle assembly, a fluid flow path, and a vortex flow adapter. The container contains a medicament which is urged out of the container by the drive mechanism. The needle assembly has a needle and/or a cannula to deliver the medicament from the container. The fluid flow path fluidically connects the container and the needle assembly. The vortex flow adapter is disposed within or defines at least a portion of the fluid flow path, and is adapted to urge the medicament to flow in a vortex pattern.

### CROSS-REFERENCE TO RELATED APPLICATIONS

Priority is claimed to U.S. Provisional Patent Application No. 62/944,083, filed Dec. 5, 2019, the entire contents of which are hereby incorporated herein by reference.

### FIELD OF DISCLOSURE

The present disclosure generally relates to drug delivery devices and, more particularly, to drug delivery devices having flow restricting assemblies to regulate drug flow.

### BACKGROUND

Drug delivery devices, such as injectors, are used to deliver liquid drugs to a patient. Upon activation, a drug delivery device may expel a drug stored within an internal reservoir of a primary container through a needle, cannula, or other delivery member into the patient. Some drug delivery devices may be temporarily attached to a patient to deliver a drug via an injection needle or some other means over an extended period of time. The drug delivery device may be adhesively attached to the tissue of the patient's abdomen, thigh, arm, or some other portion of the patient's body.

In some cases, the viscosity of a drug may vary due to a number of factors such as internal and/or external temperatures and drug concentration. The drug's viscosity may vary during a single drug administration process and may also vary among different drug delivery processes. For example, in some environments, the drug may initially have a high viscosity and thus require substantially high forces to maintain the desired flow rate, but upon the drug's viscosity decreasing due to an increase in temperature, for example, lesser forces and higher flow rates may result. In some cases, if the drug's viscosity is different than the viscosity during a previous administration process, a user may become dissatisfied upon experiencing a longer or shorter than expected drug administration, which may lead to patient uncertainty, discomfort, and/or partial dosing due to premature removal of the device by the patient.

As described in more detail below, the present disclosure sets forth systems for delivery devices embodying advantageous alternatives to existing systems and methods, and that may address one or more of the challenges or needs mentioned herein, as well as provide other benefits and advantages.

### SUMMARY

In accordance with a first aspect, a drug delivery device includes a housing, a container disposed in the housing, a drive mechanism, a needle assembly, a fluid flow path, and a vortex flow adapter. The container contains a medicament which is urged out of the container by the drive mechanism. The needle assembly has a needle and/or a cannula to deliver the medicament from the container. The fluid flow path fluidically connects the container and the needle assembly. The vortex flow adapter is disposed within or defines at least a portion of the fluid flow path and is adapted to urge the medicament to flow in a vortex pattern. In some examples, the fluid flow path may include a generally tubular member defining an interior channel. In some examples, the device may further include an activation mechanism.

In some approaches, the vortex flow adapter includes at least one chamber body and at least one disc. The at least one chamber body includes a first end having an inlet, a second end having an outlet, and a longitudinal length extending therebetween. The at least one chamber body defines a channel extending between the inlet and the outlet. The at least one disc is adapted to be disposed within the channel and includes a disc body that extends along a longitudinal length thereof. The disc body includes an outer surface and at least one groove extending along the outer surface in a first direction. The at least one groove of the disc body is adapted to urge the medicament flowing through the channel in a vortex pattern.

In some of these examples, the at least one chamber body further includes at least one vortex chamber formed by the channel. The vortex chamber may be in the form of a tapered region having a varying cross-sectional area taken along the longitudinal length of the chamber body. Further, in some examples, the chamber body includes a second vortex chamber formed by the channel.

In some examples, the drug delivery device further includes an adjacent chamber body operably coupled with the at least one chamber body. The adjacent chamber body is adapted to urge the medicament to flow in a vortex pattern having an opposite rotational flow direction than the rotational flow direction of the at least one chamber body. In these and other examples, the vortex flow adapter is adapted to generate substantial minor head losses within the fluid flow.

In accordance with a second aspect, a modular vortex flow adapter for use in a drug delivery device includes at least one chamber body and at least one disc. The at least one chamber body includes a first end having an inlet, a second end having an outlet, and a longitudinal length extending therebetween. The at least one chamber body defines a channel extending between the inlet and the outlet. The at least one disc is adapted to be disposed within the channel and includes a disc body extending along a longitudinal length. The disc body includes an outer surface and at least one groove extending along the outer surface in a first direction. The at least one groove of the disc body is adapted to urge the medicament flowing through the channel in a vortex pattern.

In accordance with a third aspect, a drug delivery device includes a housing, a container disposed in the housing, a drive mechanism, a needle assembly, a fluid flow path, and a vortex flow adapter. The container contains a medicament which is urged out of the container by the drive mechanism. The needle assembly has a needle and/or a cannula to deliver the medicament from the container. The fluid flow path fluidly connects the container and the needle assembly. The vortex flow adapter is disposed within or defines at least a portion of the fluid flow path and is adapted to generate substantial minor head losses within the fluid flow.

### BRIEF DESCRIPTION OF THE DRAWINGS

The above needs are at least partially met through provision of the flow restrictor for a drug delivery device described in the following detailed description, particularly when studied in conjunction with the drawings, wherein:

The accompanying figures show embodiments according to the disclosure and are exemplary rather than limiting.

FIG. 1 illustrates a schematic cross-sectional view of an embodiment of a drug delivery device in accordance with various embodiments;

FIG. 2 illustrates an orthographic view of an example vortex flow adapter for use with the drug delivery device of FIG. 1 in accordance with various embodiments;

FIG. 3 illustrates a close-up orthographic view of the example vortex flow adapter of FIG. 2 in accordance with various embodiments;

FIG. 4 illustrates an exploded close-up orthographic view of the example vortex flow adapter of FIGS. 2 and 3 in accordance with various embodiments;

FIG. 5 illustrates a partially-transparent side view of a second embodiment of an example flow adapter in accordance with various embodiments;

FIG. 6 illustrates an orthographic view of a third embodiment of an example flow adapter in accordance with various embodiments;

FIG. 7 illustrates an exploded orthographic view of the example flow adapter of FIG. 6 in accordance with various embodiments;

FIG. 8 illustrates a close-up orthographic view of the example flow adapter of FIGS. 6 and 7 in accordance with various embodiments;

FIG. 9 illustrates an orthographic view of a fourth embodiment of an example flow adapter in accordance with various embodiments;

FIG. 10 illustrates an orthographic view of a fifth embodiment of an example flow adapter in accordance with various embodiments;

FIG. 11 illustrates an orthographic view of a sixth embodiment of an example flow adapter in accordance with various embodiments; and

FIG. 12 illustrates a cross-sectional perspective view of a seventh embodiment of an example flow adapter in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

The present disclosure generally relates to a flow adapter for a drug delivery device. Generally, the drug delivery device includes a housing, a container, a drive mechanism, a needle assembly having first and second ends, a fluid flow connection, and a flow adapter, each of which is at least partially disposed within the housing. The container has first and second ends and contains a medicament to be administered to a user. The drive mechanism is adapted to exert a force on the first end of the container to urge the medicament through the container towards the second end thereof. The fluid flow path is coupled to the second end of the container and the first end of the needle assembly and is adapted to allow the medicament to flow from the container to the needle assembly.

The vortex flow adapter is a fluid path element that reduces the sensitivity of a drug delivery system to changes in drug viscosity. For a given operating pressure, the vortex flow adapter reduces the effect of changes in the drug viscosity on injection time. More specifically, the vortex flow adapter accomplishes this by generating substantial minor head losses within the fluid flow by generating two types of vorticity within the adapter. The vortex flow adapter generates a large-scale vorticity (spin) in alternating directions (clockwise and counter-clockwise), while also generating small-scale vorticity (i.e., turbulence) within the flow.

Because the flow adapter is designed to generate substantial minor head losses, it causes a pressure drop in the fluid flowing across the flow adapter that depends on the flow rate but does not depend explicitly on fluid dynamic viscosity. As a result, the pressure drop through the flow adapter regulates the flow rate by reducing the drive pressure by a factor that is directly proportional to the square of the flow velocity. In this manner, the flow adapter serves as a “reserve” of pressure which is available in the event of an unexpected increase in resistive pressure of the tissue and/or fluid path to complete the injection at a lower rate. Because its operation depends primarily on minor losses (as opposed to major losses which are proportional to viscosity), the vortex flow adapter provides reduced variability in injection rates across a wide range of viscosities. Stated differently, the vortex flow adapter reduces the sensitivity of the injection time of the drug delivery device to changes in drug viscosity. As a result, the flow adapter may eliminate the need for expensive electromechanical drive systems and/or closed loop feedback controls and/or systems to compensate for variations in drug viscosity.

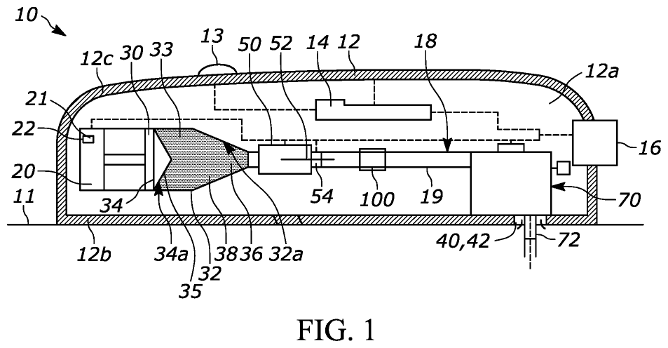

Referring to the Figures, a general drug delivery device 10 is provided that may include any number of aspects of the flow restrictor arrangement herein described. In some embodiments, including the one illustrated in FIG. 1, the drug delivery device 10 may be configured as a wearable drug delivery device, such as an on-body injector or an ambulatory infusion pump, that may be releasably coupled with a patient (e.g., to a patient's tissue 11 such as the patient's skin). In other embodiments, the drug delivery device 10 may be in the form of an autoinjector, a pen injector, or any other type of handheld devices including hybrids thereof. The drug delivery device 10 may be operated to subcutaneously or transdermally deliver a drug to a patient. The drug delivery device 10 may be configured to automatically deliver a fixed or a patient/operator-settable dose of a drug over a fixed and/or a patient/operator-settable period of time. The drug delivery device 10 may be intended for self-administration by the patient, and in some examples, or alternatively, may be used by a caregiver or a formally trained healthcare provider to administer an injection.

The drug delivery device 10 has a housing 12 that is releasably coupled with the patient's tissue 11 and having an inner volume 12a, a drive mechanism 20, a container 30, a needle assembly 70, a fluid flow connection 18 defining a sterile fluid flow path 19 between the container 30 and the needle assembly 70, and a vortex flow adapter 100, each of which may be at least partially disposed within the housing 12. It is appreciated that the releasable coupling between the housing 12 and the patient's tissue 11 can include any coupling or couplings that allow the drug delivery device 10 to be selectively secured to the patient, including the user holding the device 10 against the injection site, a suction force, an adhesive, or other means of holding the device 10 to the patient such as, for example, a strap, a clamp, and/or a bandage. Further, the drug delivery device may include an activation mechanism that includes a controller 14 and an actuator 16 (e.g., a depressible button) that is arranged on an exterior of the housing 12.

The container 30 (which, in some examples, may be referred to as a primary container) has a wall 32 that includes an interior surface 32a defining an interior volume 33 that accommodates a plunger 34. The plunger 34 is moveably disposed within the container 30 and has a first end 34a that includes an interior surface 35. The interior surface 32a of the container 30 and the interior surface 35 of the plunger 34 define a reservoir 36 that contains a drug or medicament 38.

The volume of the drug 38 contained in the reservoir 36 prior to delivery may be: any volume in a range between approximately (e.g., ±10%) 0.5-20 mL, or any volume in a range between approximately (e.g., ±10%) 0.5-10 mL, or any volume in a range between approximately (e.g., ±10%) 1-10 mL, or any volume in a range between approximately (e.g., ±10%) 1-8 mL, or any volume in a range between approximately (e.g., ±10%) 1-5 mL, or any volume in a range between approximately (e.g., +10%) 1-3.5 mL, or any volume in a range between approximately (e.g., ±10%) 1-3 mL, or any volume in a range between approximately (e.g., ±10%) 1-2.5 mL, or any volume in a range between approximately (e.g., +10%) 1-2 mL, or any volume equal to or less than approximately (e.g., ±10%) 4 mL, or any volume equal to or less than approximately (e.g., ±10%) 3.5 mL, or any volume equal to or less than approximately (e.g., ±10%) 3 mL, or any volume equal to or less than approximately (e.g., ±10%) 2.5 mL, or any volume equal to or less than approximately (e.g., ±10%) 2 mL, or any volume equal to or less than approximately (e.g., ±10%) 1.5 mL, or any volume equal to or less than approximately (e.g., ±10%) 1 mL, or any volume equal to or greater than approximately (e.g., ±10%) 2 mL, or any volume equal to or greater than approximately (e.g., ±10%) 2.5 mL, or any volume equal to or greater than approximately (e.g., ±10%) 3 mL. The reservoir may be completely or partially filled with the drug or medicament 38. The drug or medicament 38 may be one or more of the drugs listed below such as, for example, a granulocyte colony-stimulating factor (G-CSF), a PCSK9 (Proprotein Convertase Subtilisin/Kexin Type 9) specific antibody, a sclerostin antibody, or a calcitonin gene-related peptide (CGRP) antibody.

The housing 12 may include a bottom wall 12b to contact or to be releasably coupled (e.g., adhered with an adhesive) with the patient's skin 11, and a top wall 12c including one or more visual feedback mechanisms 13 such as, for example a window, an opening, and/or an illumination system (not illustrated) for viewing the container 30 and the drug or medicament 38 contained therein. The one or more visual feedback mechanisms 13 may also be used to communicate information to the user about the operational state of the drug delivery device 10 and/or the condition of the drug or medicament 38. An opening 40 may be formed in the bottom wall 12b, and optionally a pierceable sterile barrier or septum 42 may extend across the opening 40 to seal the interior 12a of the housing 12 prior to use. In some embodiments, the pierceable sterile barrier 42 may be omitted, and instead a removable sealing member (not illustrated) may cover and seal the opening 40 prior to use. The exterior of the needle assembly 70 may be defined by a housing (not illustrated) that is separate from the drug delivery device housing 12.

The fluid flow connection 18 connects the container 30, and more specifically the reservoir 36, to the needle assembly 70. The actuator 16 is configured to initiate operation of the drug delivery device 10 by activating, via mechanical and/or electrical means (shown in dotted lines in FIG. 1), the drive mechanism 20, the needle assembly 70, the controller 14, and/or other mechanisms and/or electronics. In some examples, wireless communication may be employed to cause the drug delivery device 10 to be activated. In embodiments where the actuator 16 is a button that is depressed or otherwise physically moved by a user or patient, the actuator 16 may be configured to exert a motive force and/or transmit a signal needed to activate the needle assembly 70, the fluid flow connection 18, the drive mechanism 20, the controller 14, and/or other mechanisms. In such embodiments, the actuator 16 may be physically connected to, either directly or indirectly via a mechanical linkage, the needle assembly 70, the drive mechanism 20, the fluid flow connection 18, and/or other mechanisms such that manually depressing or otherwise interacting with the actuator 16 supplies the motive force necessary to activate the needle assembly 70, the drive mechanism 20, the fluid flow connection 18, and/or other mechanisms.

As previously noted, the fluid flow connection 18 defines a sterile fluid flow path 19 between the container 30 and the assembly mechanism 70. The fluid flow connection 18 may be in the form of a flexible tube member defining an interior channel. In some examples, the fluid flow connection 18 may be sterilized, and may be partially or entirely made of a polymer or other material. In some examples, a container access mechanism 50 is coupled to the fluid flow connection 18 and is configured to insert a container needle 52 through a septum 54 associated with and/or covering the container 30 to establish fluid communication between the container 30 and the sterile fluid flow path 19 in response to activation of the drug delivery device 10, for example, via the actuator 16. In the illustrated examples, relative movement between the container 30 and the container access mechanism 50 causes the container needle 52 to pierce the septum 54. In some examples, the container needle 52 may be staked to the container 30 such that the container needle 52 cannot move relative to the wall 32 of the container 30; whereas, in other examples, the container needle 52 may be moveable relative to the container 30 and may access the reservoir 36 of the container 30 by piercing through the septum 54 or other sterile barrier covering an opening in the container 30 during operation or set up the drug delivery device 10. In some examples, the needle assembly 70 and the container 30 and/or other components such as the container access mechanism 50 may be integrated into a single unit, and thus the fluid flow connection 18 may not be included in the drug delivery device 10.

For example, in some embodiments, manually depressing or otherwise moving the actuator 16 may cause the fluid flow connection 18 and the container access mechanism 50 to move towards the container 30, or cause the container 30 to move towards the fluid flow connection 18 and the container access mechanism 50, and thereby cause the container needle 52 to penetrate through the seal member or septum 54, thereby fluidically connecting the reservoir 36 and the fluid flow path 19.

Additionally, or alternatively, the actuator 16 may operate as an input device that transmits an electrical, optical, and/or mechanical signal to the controller 14, which in turn may execute programmable instructions to control operation of the needle assembly 70, the drive mechanism 20, the fluid flow connection 18, and/or other mechanisms. In such embodiments, the controller 14 may include a processor (e.g., a microprocessor) and a non-transitory memory for storing the programmable instructions to be executed by the processor. Furthermore, in such embodiments, the drug delivery device 10 may include an internal actuator (e.g., an electric motor, a pneumatic or hydraulic pump, and/or a source of pressurized gas or liquid) which is separate from the actuator 16 and which, in response to a control signal received from the controller 14, exerts the motive force needed to activate the needle assembly 70, the drive mechanism 20, the container access mechanism 50, and/or other mechanisms.

The drive mechanism 20 may include any number of components and/or sub-components to drive, urge, and/or exert a force on the plunger 34 to cause the drug or medicament 38 stored therein to be dispensed therefrom and to operate the needle assembly 70. In some examples, the drive mechanism 20 may use a drive fluid 22 in the form of a compressed CO₂ gas or other compressed gas and/or a compressed liquid to drive, urge, and/or exert the force on the plunger 34. The drive fluid 22 may initially be stored within a pressure vessel or other container 21, and the drive mechanism 20 may be configured to release the compressed gas and/or liquid from the pressure vessel or other container 21 by opening a valve (not illustrated), which allows the compressed gas and/or liquid to flow into the container 30. In other examples, the drive mechanism 20 may be in the form of a hydro-pneumatic actuation system whereby a hydraulic and/or pneumatic force is exerted on the drive fluid 22 to move the plunger 34 through the container 30 to expel the drug 38 therefrom. In other examples, the drive mechanism 20 may include any number of resilient members (e.g., springs) that exert an urging force on the plunger 34. Examples of suitable activation mechanisms 20 are described in U.S. App. No. 62/543,058, filed on Aug. 9, 2017, the entire contents of which are incorporated by reference herein. Other examples of suitable activation mechanisms 20 are possible.

The needle assembly 70 may include any number of components that insert a needle and/or a cannula 72, and may include any number of systems and/or subsystems necessary to complete this task. Such systems and/or subsystems will not be discussed in further detail herein.

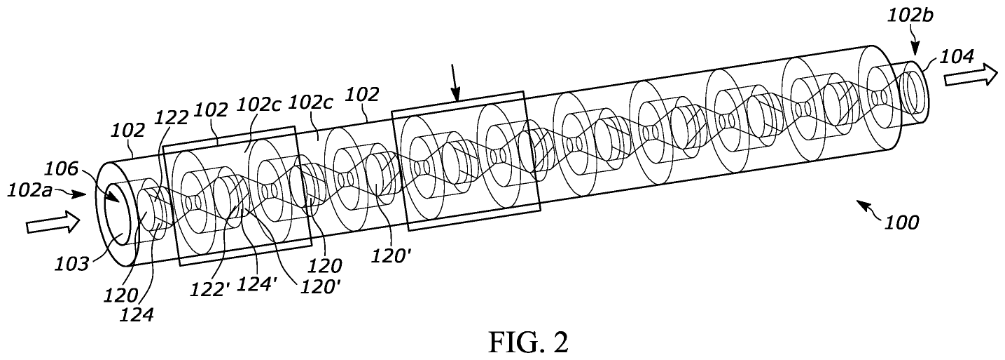

As illustrated in FIGS. 2-4, a first example vortex flow adapter 100 includes any number of chamber bodies 102, each containing at least one disc 120. Generally speaking, the vortex flow adapter 100 is in the form of a coaxial vortex flow restrictor. It will be appreciated that each of the chamber bodies 102 may include similar components and/or features, and as such, for descriptive purposes, reference is primarily made to a single chamber body 102. The chamber body 102 includes a first end 102a having an inlet 103 for receiving fluid flow in the direction of the arrow in FIG. 2, a second end 102b having an outlet 104 for fluid to exit in the direction of the arrow in FIG. 2, and a longitudinal length 102c extending therebetween. Further, the chamber body 102 defines a channel 106 extending between the inlet 103 and the outlet 104. Each of the chamber bodies 102 may have a longitudinal length 102c between approximately 2 mm and approximately 5 mm, though other dimensions are possible.

In the illustrated example, the chamber body 102 is generally cylindrical in shape. The longitudinal length 102c thereof has a substantially constant outer dimension (e.g., diameter), though in some examples, other configurations and/or shapes are possible. The second end 102b of the chamber body 102 forms a stepped region that has a reduced outer dimension (e.g., diameter) than the remainder of the longitudinal length 102c thereof. More specifically, the second end 102b of the chamber body 102 forms a coupling portion that is dimensioned to be insertable into the inlet 103 (which also forms a coupling portion) at the first end 102a of an adjacent chamber body 102. Accordingly, the chamber bodies 102 are modular in that any number of discrete chamber bodies 102 may be used to form the vortex flow adapter 100 to suit specific operating conditions such as nominal fluid viscosity and/or operating pressure. It is appreciated that in some examples, the inlet 103 and the outlet 104 directions may be reversed.

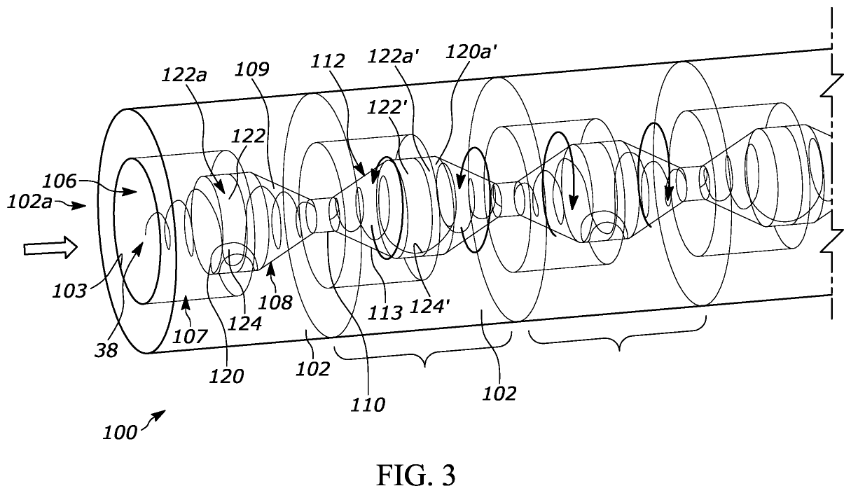

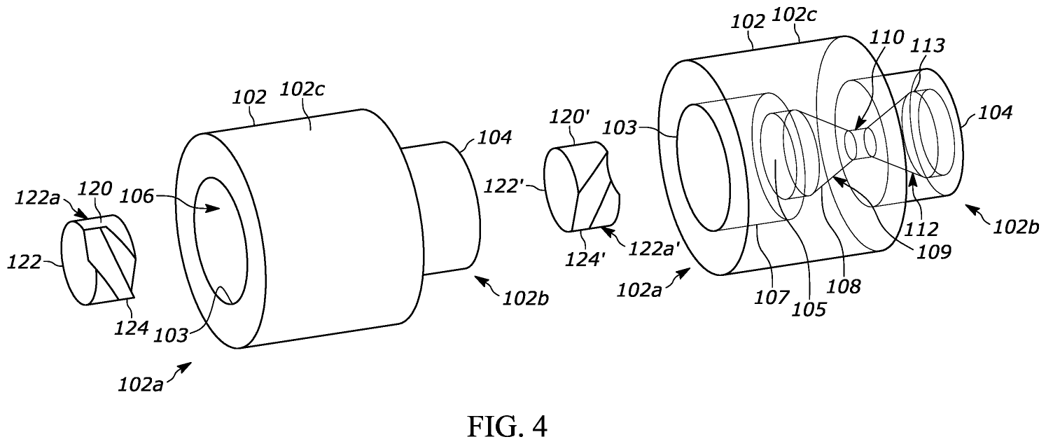

With reference to FIGS. 3 and 4, the channel 106 includes a number of regions. A first region 107 of the channel 106 is defined by the inlet 103 and is generally circular in cross-section. A second region 108 of the channel 106 is in the form of a first vortex chamber 109, and an intervening region 105 (see, FIG. 4) is cylindrical in shape and disposed between the first region 107 and the second region 108. The vortex chamber 109 is in the form of a tapered region in which the sidewall defining the channel 106 decreases in cross-sectional dimension (e.g., diameter) in the direction from the first end 102a towards the second end 102b. The channel 106 further includes an optional port 110 positioned adjacent to the second region 108, and a third region 112 in the form of a second vortex chamber 113.

The third region 112 is generally positioned at or near the outlet 104 and the second end 102b of the chamber body 102. More specifically, the second vortex chamber 113 is in the form of an oppositely-tapered region from the first vortex chamber 109. Put differently, the second vortex chamber 113 is defined by the sidewall that defines the channel 106 increasing in cross-sectional dimension (e.g., diameter) in the direction from the first end 102a towards the second end 102b. Accordingly, when viewed together, the second region 108, the port 110, and the third region 112 combine to form a generally hourglass shape.

The disc 120 includes a generally cylindrical disc body 122 that corresponds to a shape and dimension of the channel 106. The disc body 122 has an outer surface 122a and an elongated groove 124 extending along the outer surface 122a. It is understood that any number of desired shapes that correspond to the shape of the channel 106 may be used. The groove 124 is formed into the disc body 122 such that an exterior channel is formed. In the illustrated example, the groove 124 is a helical groove that extends along the outer surface 122a in a first direction. As illustrated in FIG. 4, a second disc 120′ is provided that includes similar features to the disc 120, and as such, these features are designated by similar reference characters as the disc 120 appended by a prime (“′”). These similar features will not be discussed in substantial detail. However, the second disc 120′ differs from the first disc 120 in that the groove 124′ of the second disc 120′ extends in an opposite direction than the groove 124 of the first disc 120. More specifically, the groove 124 in the first disc 120 will cause the drug or medicament 38 to flow in a vortex pattern in a clockwise direction when flowing through the channel 106, and the groove 124′ in the second disc 120′ will cause the drug or medicament 38 to flow in a vortex pattern in a counter-clockwise direction when flowing through the channel 106.

The disc 120 (and/or the second disc 120′) is disposed within the channel 106. More specifically, the disc 120 (and/or the second disc 120′) is positioned in the first region 107 of the channel 106, including a portion of the disc 12 (and/or the second disc′) being positioned at least partially in the intervening region 105. In the illustrated example, a number of chamber bodies 102 are coupled together by inserting the second end 102b of one chamber body 102 into the first end 102a of an adjacent chamber body 102. In the illustrated example, the chamber bodies 102 alternate between first and second discs 120, 120′. More specifically, a first chamber body 102 has a first disc 120 disposed in the channel 106, and an adjacent chamber body 102 has a second disc 120′ disposed in the channel 106. Any combination of first and/or second discs 120, 120′ may be placed in any number of adjacent chamber bodies 102 as desired.

As previously noted, in some examples, the vortex flow adapter 100 is dimensioned to be disposed within the fluid flow path 19. In other examples, the vortex flow adapter is configured to be coupled to discrete segments of the fluid flow path 19. In these examples, a first portion of the fluid flow path 19 is coupled to an inlet 102a of the chamber body 102, and a second portion of the fluid flow path 19 is coupled to an outlet 102b of a chamber body 102. In any of these configurations, the drug or medicament 38 flows through the vortex flow adapter 100 prior to reaching the needle assembly 70. As illustrated in FIG. 3 and FIG. 4, upon activating the device 10, the drug 38 enters the inlet 103 of a chamber body 102 and the first region 107 of the channel 106. The drug 38 then enters the groove 124 of the disc 120 and is urged towards the second region 108 of the channel 106. The configuration of the groove 124 urges the drug 38 through the channel 106 in a generally clockwise vortex pattern.

Upon exiting the disc 120, the drug or medicament 38 enters the second region 108 of the channel 106 in the generally clockwise direction, through the port 110, and into the third region 112 that includes the second vortex chamber 113. The rapid changes in cross-sectional area between the first vortex chamber 109, the port 110, and the second vortex chamber 113 create additional turbulence within the fluid flow. The drug or medicament 38 then enters the groove 124′ of the second disc 120′ which causes the drug or medicament 38 to flow in a generally counter-clockwise vortex pattern. In this manner, the drug or medicament 38 continues to flow through the adjacent chamber bodies 102, in alternating vortex patterns.

The changing cross-sectional areas of the first and the second vortex chambers 109, 113, combined with the alternating vortex patterns caused by the discs 120, 120′, together create significant minor head losses within the fluid flow. As a result, the device 10 produces consistent and predictable injection rates across a wide range of operating conditions when delivering the drug or medicament 38.

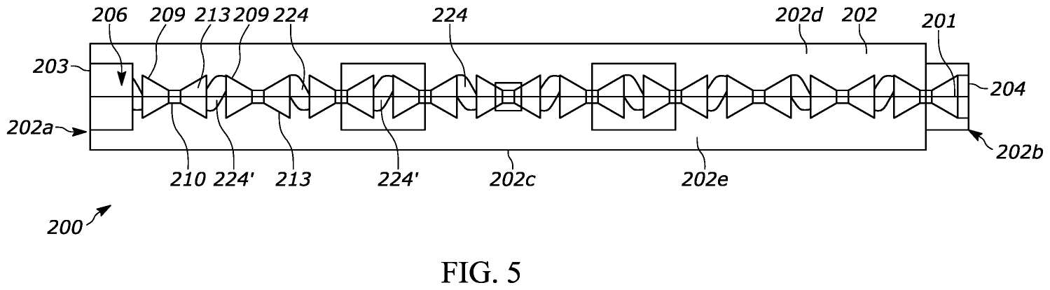

A second embodiment of a vortex flow adapter 200 coupled with a fluid flow path 19 is illustrated in FIG. 5. It will be appreciated that the vortex flow adapter 200 illustrated in FIG. 5 may include similar features to the vortex flow adapter 100, and accordingly, elements illustrated in FIG. 5 are designated by similar reference numbers indicated in the embodiment illustrated in FIGS. 1-4 increased by 100. Accordingly, these features will not be described in substantial detail. Further, it is appreciated that any of the elements described with regards to the vortex flow adapter 100 may be incorporated into the vortex flow adapter 200.

In this embodiment, the vortex flow adapter 200 includes a single, elongated chamber body 202 that is defined by an upper body 202d and a lower body 202e that are operably coupled together. Such an elongated chamber body 202 may be constructed via an injection molding process which may form the channel 206 and alternating vortex chambers 209, 213. Further, in this example, the chamber body 202 includes helical portions 224, 224′ that replace the grooves formed in the discs used in the vortex flow adapter 100. These helical portions 224, 224′ urge the drug or medicament 38 to flow in alternating vortex patterns. In some examples, the vortex flow adapter 200 additionally includes a seal 201 positioned between the upper and the lower body 202d, 202e to prevent the drug or medicament 38 from leaking.

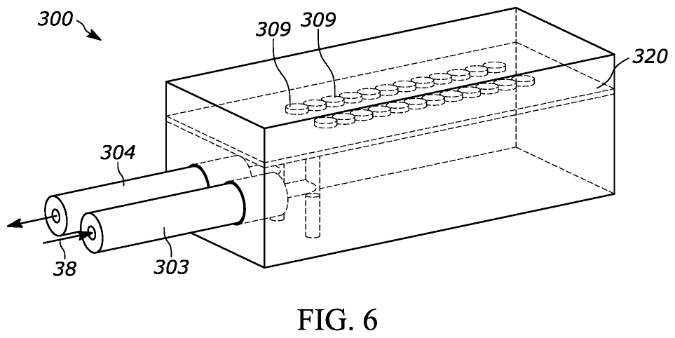

A third embodiment of a vortex flow adapter 300 coupled with a fluid flow path 19 is illustrated in FIGS. 6-8. It will be appreciated that the vortex flow adapter 300 illustrated in FIGS. 6-8 may include similar features to the vortex flow adapters 100, 200, and accordingly, elements illustrated in FIGS. 6-8 are designated by similar reference numbers indicated in the embodiments illustrated in FIGS. 1-5 increased by 100 and 200, respectively. Accordingly, these features will not be described in substantial detail. Further, it is appreciated that any of the elements described with regards to the vortex flow adapters 100, 200 may be incorporated into the vortex flow adapter 300.

In this example, the vortex flow adapter 300 is in the form of an offset vortex flow adapter that includes a body 302 having an upper body 302a and a lower body 302b which are separated by a separator port plate 320 having a number of ports 320a. In some examples, alignment pins 318 are used to align the upper and lower bodies 302a, 302b. The lower body 302b includes an inlet tube 303 and an outlet tube 304. Further, the vortex flow adapter 300 includes any number of vortex chambers 309, 313 as desired to accommodate specific operating conditions such as nominal fluid viscosity or operating pressure. The inlet tube 303 is operably coupled with a first vortex chamber 313.

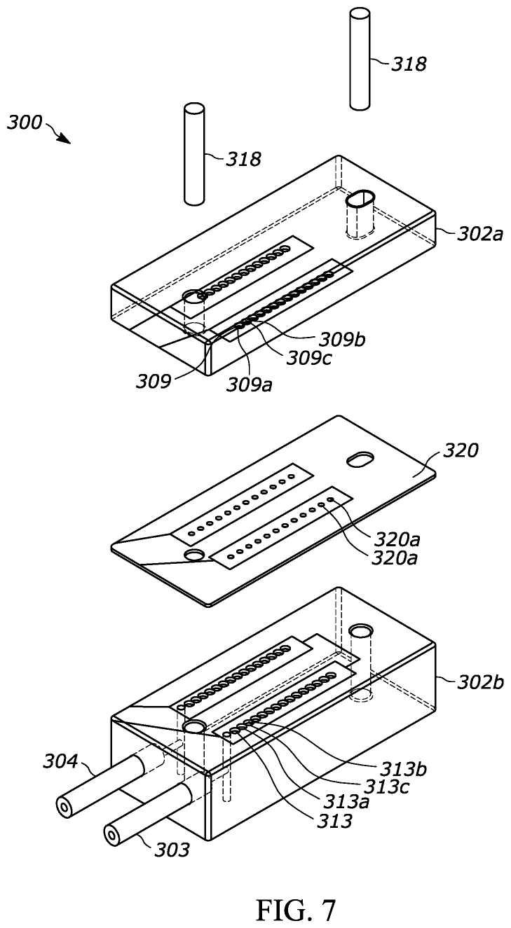

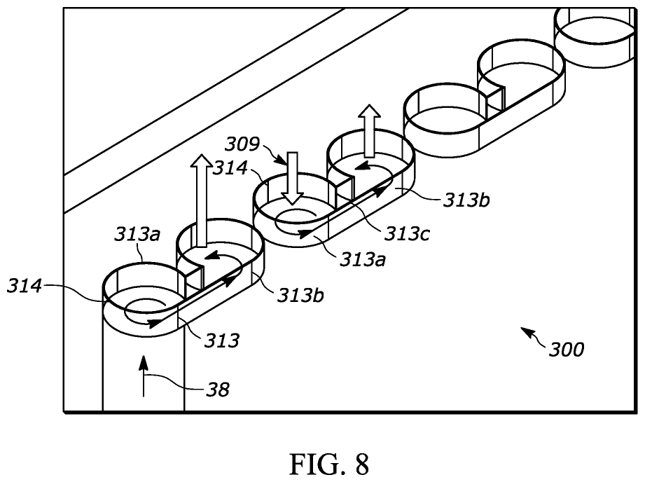

As illustrated in FIG. 8, the drug or medicament 38 enters a vortex chamber 313 of the lower body 302b having a first section 313a and a second section 313b fluidically coupled together via a side port 313c, each of which has a similar corresponding sidewall 314. More specifically, the drug or medicament 38 enters into the first section 313a, and is then urged in a counter-clockwise vortex pattern tangentially through the side port 313c into the second section 313b, where the drug or medicament 38 flows in a counter-clockwise vortex pattern and up through a port 320a (shown in FIG. 7) of the separator port plate 320 (shown in FIG. 7).

While not illustrated, the drug or medicament 38 then enters a corresponding first section 309a of the next vortex chamber 309 in the upper body 302a and is urged in a clockwise vortex pattern tangentially through a side port 309c and into a second section 309b of the vortex chamber 309, whereupon the drug or medicament 38 continues to flow in a clockwise vortex pattern downwards through the next port 320a of the separator port plate 320. Such alternating upwards and downwards flow, which also alternates in clockwise and counter-clockwise vortex directions, continues until the drug or medicament 38 exits through the outlet tube 304 to be delivered via the needle assembly 70.

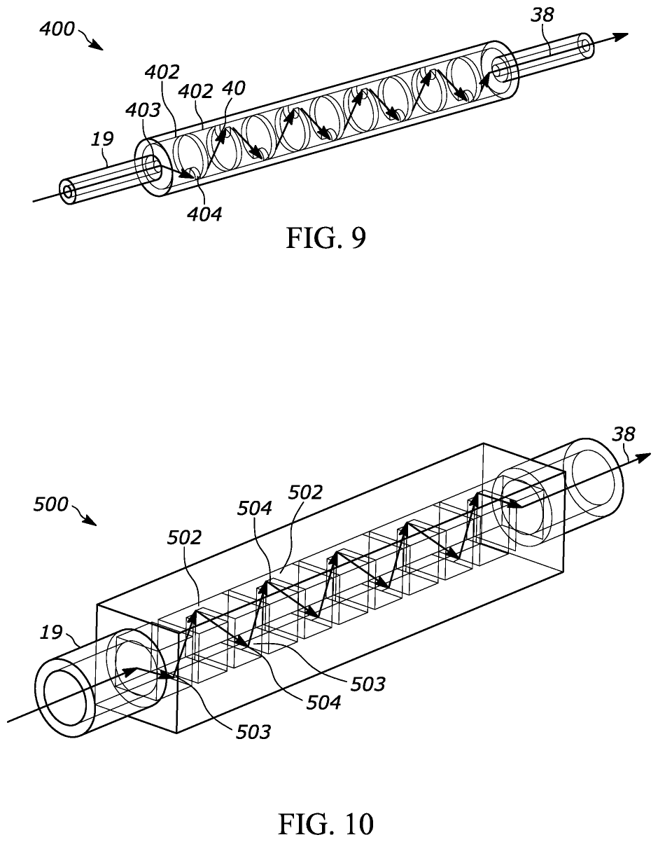

A fourth embodiment of a vortex flow adapter 400 coupled with a fluid flow path 19 is illustrated in FIG. 9. It will be appreciated that the vortex flow adapter 400 illustrated in FIG. 9 may include similar features to the vortex flow adapters 100, 200, and 300, and accordingly, elements illustrated in FIG. 9 are designated by similar reference numbers indicated in the embodiments illustrated in FIGS. 1-8 increased by 100, 200, and 300, respectively. Accordingly, these features will not be described in substantial detail. Further, it is appreciated that any of the elements described with regards to the vortex flow adapters 100, 200, 300 may be incorporated into the vortex flow adapter 400.

In this example, the vortex flow adapter 400 is in the form of a number of cylindrical chambers 402 connected to each other in series. In this embodiment, the entrance and exit ports 403, 404 of each chamber are located along a diagonal of the circular cross-section of the chamber. The drug 38 enters the chamber at a high velocity and approximately axial direction but decelerates and deflects under the developed pressure gradient within the chamber 402. Such an arrangement may assist in maximizing the minor head loss in each chamber 402 while reducing chamber length. This may additionally increase the turbulence and local vortices when compared to similar sized orifices while achieving the same or near minor head loss results while using a shorter chamber.

A fifth embodiment of a vortex flow adapter 500 coupled with a fluid flow path 19 is illustrated in FIG. 10. It will be appreciated that the vortex flow adapter 500 illustrated in FIG. 10 may include similar features to the vortex flow adapters 100, 200, 300, and 400, and accordingly, elements illustrated in FIG. 10 are designated by similar reference numbers indicated in the embodiments illustrated in FIGS. 1-9 increased by 100, 200, 300, and 400, respectively. Accordingly, these features will not be described in substantial detail. Further, it is appreciated that any of the elements described with regards to the vortex flow adapters 100, 200, 300, 400 may be incorporated into the vortex flow adapter 500.

In this example, the vortex flow adapter 500 is in the form of a number of generally cuboid (rectangular) chambers 502 connected to each other in series. As with the fourth embodiment 400, in this embodiment, the entrance and exit ports 503, 504 of each chamber are located along a diagonal of the rectangular cross-section of the chamber. The drug 38 enters the chamber at a high velocity and approximately axial direction but decelerates and deflects under the developed pressure gradient within the chamber 502. Such an arrangement may assist in maximizing the minor head loss in each chamber 502 while reducing chamber length. This may additionally increase the turbulence and local vortices when compared to similar sized orifices while achieving the same or near minor head loss results while using a shorter chamber. By using cuboid chambers 502, the pressure distribution, and thus the pressure drop, within the chamber is different than what is experienced in the pressure chamber 400.

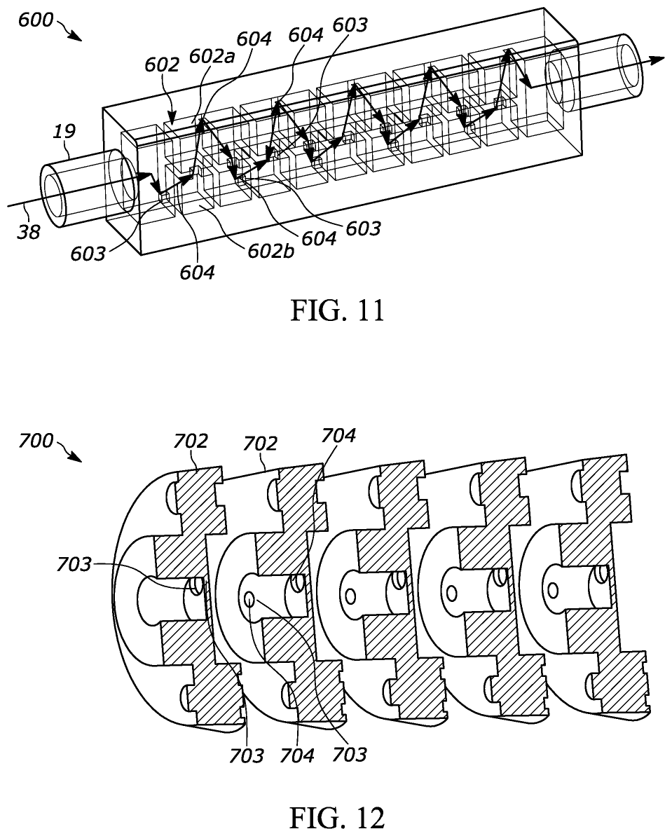

A sixth embodiment of a vortex flow adapter 600 coupled with a fluid flow path 19 is illustrated in FIG. 11. It will be appreciated that the vortex flow adapter 600 illustrated in FIG. 11 may include similar features to the vortex flow adapters 100, 200, 300, 400, and 500, and accordingly, elements illustrated in FIG. 11 are designated by similar reference numbers indicated in the embodiments illustrated in FIGS. 1-10 increased by 100, 200, 300, 400, and 500, respectively. Accordingly, these features will not be described in substantial detail. Further, it is appreciated that any of the elements described with regards to the vortex flow adapters 100, 200, 300, 400, 500 may be incorporated into the vortex flow adapter 600.

In this example, the vortex flow adapter 600 is in the form of a number of generally cuboid (rectangular) chambers 602 connected to each other in series. Notably, the adapter 600 uses two or more rows of chambers 602a, 602b to enable packaging a greater number of chambers therein without increasing the length of the adapter 600. The upper and lower chambers 602a, 602b are coupled with each other via a port located at or near a middle of the axial edge on opposite sides of the adjacent chamber port. Such an orientation of the entrance and exit ports 603, 604 of each chamber provides a larger space for generation of potential vortices inside the chamber 602.

A seventh embodiment of a vortex flow adapter 700 coupled with a fluid flow path 19 is illustrated in FIG. 12. It will be appreciated that the vortex flow adapter 700 illustrated in FIG. 12 may include similar features to the vortex flow adapters 100, 200, 300, 400, 500, and 600, and accordingly, elements illustrated in FIG. 12 are designated by similar reference numbers indicated in the embodiments illustrated in FIGS. 1-11 increased by 100, 200, 300, 400, 500, and 600, respectively. Accordingly, these features will not be described in substantial detail. Further, it is appreciated that any of the elements described with regards to the vortex flow adapters 100, 200, 300, 400, 500, 600 may be incorporated into the vortex flow adapter 700.

In this example, the vortex flow adapter 700 is similar to the vortex flow adapter 400 but is in the form of modular disks 702 that may be coupled with each other via a press-fit connection. The entrance and exit ports 703, 704 of each disk 702 are located along a diagonal of the circular cross-section of the disk 702. The drug 38 enters the disk 702 at a high velocity and approximately axial direction, but decelerates and deflects under the developed pressure gradient within the disk 702.

So configured, the flow adapters described herein cause the drug to be urged in alternating vortex patterns through the vortex chambers thereby generating the optimal level of minor head losses to reduce the variability in fluid injection rates. The flow adapters may be beneficial in limiting changes to injection rates caused by changes in drug viscosity. Further, in examples where the injector is in the form of a pen-type or handheld injector, reduced variability of flow rates can reduce occurrences of the patient misjudging injection times and prematurely removing the device. Additionally, the flow adapter can be implemented in handheld devices in a cost-effective manner, since these devices may not contain complex electromechanical drive systems with feedback to compensate for variations in viscosity or operating conditions.

As illustrated in the below table, example flow rates through the flow restrictor were measured at different inlet pressures between approximately 20 psi to approximately 85 psi for two different sample fluids having different viscosities. The first fluid had a viscosity of approximately 1 cP, and the second fluid had a viscosity of approximately 61 cP. While pressure drops vary quadratically with the flow rate for both fluids, it has a minor dependence on viscosity. A factor of approximately 60 times an increase in viscosity can change the pressure drop across the flow restrictor (at the same flow rate) by less than 20%.

TABLE 1
Measured flow rates through flow
controller at varying pressures for water
Water at ~1 cP

| Inlet pressure (psi) | Average flow rate (ml/sec) |
|---|---|
| 19.5 | 1.35 |
| 36.5 | 1.82 |
| 55 | 2.23 |
| 75.5 | 2.58 |
| 85 | 2.79 flow |

TABLE 2
Measured flow rates through flow controller
at varying pressures for Glycerol
Glycerol at ~60 cP

| Inlet pressure (psi) | Average flow rate (ml/sec) |
|---|---|
| 21.5 | 1.14 |
| 36 | 1.53 |
| 55 | 1.95 |
| 76 | 2.33 |
| 84 | 2.41 |

A combination of major and minor loss models were fitted to this data. The coefficient of minor loss for the element is approximately 18, the inner volume of the flow controller is approximately 0.056 mL, and the major loss at the worst case (e.g., 60 cP upper band of the viscosity range) is still less than approximately 20% of the total pressure drop.

It is recognized that the vortex flow adapters described herein may include any number of modifications and/or alternatives. For example, in any of the vortex flow adapters, the ports and/or separator plates may be omitted. Further, in some examples, the vortex chambers may be conical, cylindrical, or any other shape or combination of shapes.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-a4B7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP lib/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-a5R1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Rα mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(18)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA) x anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3) x anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP x 4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vllI (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA) x anti-CD3 BiTE® (bispecific T cell engager) construct. In some embodiments, the drug delivery device may contain or be used with AMG 757 or another product containing a half-life extended (HLE) anti-delta-like ligand 3 (DLL3) x anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a housing defining an inner volume;
a container at least partially disposed within the housing, the container containing a medicament;
a drive mechanism at least partially disposed within the housing, the drive mechanism adapted to exert a force to urge the medicament out the container;
a needle assembly having a needle and/or a cannula to deliver the medicament;
a fluid flow path fluidically connecting the container and the needle assembly; and
a vortex flow adapter disposed within or defining at least a portion of the fluid flow path, the vortex flow adapter adapted to urge the medicament to flow in a vortex pattern.

2. The drug delivery device of claim 1, wherein the vortex flow adapter comprises:
at least one chamber body including a first end having an inlet, a second end having an outlet, and a longitudinal length extending therebetween, the at least one chamber body defining a channel extending between the inlet and the outlet; and
at least one disc adapted to be disposed within the channel, the at least one disc including a disc body extending along a longitudinal length, the disc body including an outer surface and at least one groove extending along the outer surface in a first direction;
wherein the at least one groove of the disc body is adapted to urge the medicament flowing through the channel in the vortex pattern.

3. The drug delivery device of claim 2, wherein the at least one chamber body further includes at least one vortex chamber formed by the channel.

4. The drug delivery device of claim 3, wherein the at least one vortex chamber comprises a tapered region having a varying cross-sectional area along the longitudinal length of the chamber body.

5. The drug delivery device of claim 3, wherein the at least one chamber body further comprises a second vortex chamber formed by the channel.

6. The drug delivery device of claim 1, further comprising an adjacent chamber body operably coupled with the at least one chamber body, the adjacent chamber body adapted to urge the medicament to flow in a vortex pattern having an opposite rotational flow direction than a rotational flow direction of the at least one chamber body.

7. The drug delivery device of claim 1, wherein the vortex flow adapter is adapted to generate a minor head loss to the medicament flowing within the fluid flow path.

8. The drug delivery device of claim 1, wherein the fluid flow path comprises a generally tubular member defining an interior channel.

9. A modular vortex flow adapter for use in a drug delivery device, the modular vortex flow adapter comprising:
at least one chamber body including a first end having an inlet, a second end having an outlet, and a longitudinal length extending therebetween, the at least one chamber body defining a channel extending between the inlet and the outlet; and
at least one disc adapted to be disposed within the channel, the at least one disc including a disc body extending along a longitudinal length, the disc body including an outer surface and at least one groove extending along the outer surface in a first direction;
wherein the at least one groove of the disc body is adapted to urge the medicament flowing through the channel in a vortex pattern.

10. The modular vortex flow adapter of claim 9, wherein the at least one chamber body further includes at least one vortex chamber formed by the channel.

11. The modular vortex flow adapter of claim 10, wherein the at least one vortex chamber comprises a tapered region having a varying cross-sectional area along the longitudinal length of the chamber body.

12. The modular vortex flow adapter of claim 10, wherein the at least one chamber body further comprises a second vortex chamber formed by the channel.

13. The modular vortex flow adapter of claim 9, wherein the at least one chamber body further comprises a first coupling portion at the first end and a second coupling portion at the second end, wherein the first and the second coupling portions are adapted to couple with an additional chamber body.

14. A drug delivery device comprising:
a housing defining an inner volume;
a container at least partially disposed within the housing, the container containing a medicament;
a drive mechanism at least partially disposed within the housing, the drive mechanism adapted to exert a force to urge the medicament out the container;
a needle assembly having a needle and/or a cannula to deliver the medicament;
a fluid flow path fluidically connecting the container and the needle assembly; and
a vortex flow adapter disposed within or defining at least a portion of the fluid flow path, the vortex flow adapter adapted to generate a minor head loss to the medicament flowing within the fluid flow path.

15. The drug delivery device of claim 14, wherein the vortex flow adapter comprises:
at least one chamber body including a first end having an inlet, a second end having an outlet, and a longitudinal length extending therebetween, the at least one chamber body defining a channel extending between the inlet and the outlet; and
at least one disc adapted to be disposed within the channel, the at least one disc including a disc body extending along a longitudinal length, the disc body including an outer surface and at least one groove extending along the outer surface in a first direction;
wherein the at least one groove of the disc body is adapted to urge the medicament flowing through the channel in a vortex pattern.

16. The drug delivery device of claim 15, wherein the at least one chamber body further includes at least one vortex chamber formed by the channel.

17. The drug delivery device of claim 16, wherein the at least one vortex chamber comprises a tapered region having a varying cross-sectional area along the longitudinal length of the chamber body.

18. The drug delivery device of claim 16, wherein the at least one chamber body further comprises a second vortex chamber formed by the channel.

19. The drug delivery device of claim 14, wherein the at least one chamber body further comprises a first coupling portion at the first end and a second coupling portion at the second end, wherein the first and the second coupling portions are adapted to couple with an additional chamber body.

20. The drug delivery device of claim 14, wherein the fluid flow path comprises a generally tubular member defining an interior channel.
